(19) United States
(12) Patent Application Publication
(10) Pub. No.: US 2026/0355058 A1
(43) Pub. Date: Nov. 12, 2026

(54) Closed-System Drug Transfer Devices With Controlled Connect and Disconnect Features

(71) Applicant: Becton, Dickinson and Company, Franklin Lakes, NJ (US)

(72) Inventors: Rafael Gonzalez Macias, San Diego, CA (US); Rahul Rajan Puthukkad, Bengaluru (IN); Mohammed Mehtab Khan, Bengaluru (IN); Manjunath Mangumali, Bangalore (IN); Prashant Dalavi, Belgaum (IN); Shwetha Mahadevappa, Phoenix, AZ (US); Kaushik Suman, Jharkhand (IN); Sanskriti Jain, Shahdara (IN); Nakul Sudarshan, Bengaluru (IN)

(21) Appl. No.: 19/202,397

(22) Filed: May 8, 2025

(51) Int. Cl. A61J 1/20; A61J 1/14

(52) U.S. Cl. CPC A61J 1/2055; A61J 1/1406; A61J 1/201

(57) ABSTRACT

Provided herein is a closed system drug transfer device. The closed system drug transfer device comprises an injector and a connector. The injector includes a body, a luer hub comprising a female luer connection, and a cannula. The connector is configured to be inserted into the injector through the distal end of the body. The connector includes a central shaft defining a lumen and a seal positioned in a proximal opening of the central shaft, the seal being pierceable by the cannula when the connector is inserted into the injector, to fluidly connect the cannula to the lumen. The closed system drug transfer device includes controlled connect and disconnect features configured to prevent accidental disconnection between the connector and the injector and/or prevent accidental or intentional disconnection between the injector and the male luer connection of a fluid source, container, or IV line.

### BACKGROUND OF THE INVENTION

### Field of the Invention

The present disclosure relates generally to closed-system drug transfer devices and, more specifically, to features of closed-system drug transfer devices that provide for controlled connection and disconnection of components thereof.

### Description of Related Art

Healthcare providers and medical device and pharmaceutical manufacturers, as well as, leading governmental and regulatory agencies, have long recognized the issues healthcare workers face with compounding and administering hazardous drugs. For example, nurses treating cancer patients risk being exposed to chemotherapy drugs when compounding and administering these drugs, and this unintentional chemotherapy exposure can affect the nervous system, impair the reproductive system, and/or bring an increased risk of developing blood cancers in the future.

In order to reduce the risk of healthcare workers being exposed to hazardous drugs, the closed transfer of these drugs becomes important, with various policies and regulations having been implemented that require the use of closed-system drug transfer devices (CSTDs) when compounding and administering hazardous drugs. A CSTD may provide for the transfer of hazardous drugs out from, between, or through syringes, vials, intravenous (IV) fluid bags, and IV lines, as non-limiting examples, with the CSTD providing for a safe removal, transfer, and/or administration of the hazardous drug. The CSTD may utilize a physical barrier that inhibits the escape of the hazardous drug substance to thereby mitigate the exposure risk to the healthcare worker who may compound or administer hazardous drugs to patients.

A typical CSTD as known in the art may include a fluid container injector, such as a BD PhaSeal™ Optima Injector from Becton, Dickinson and Company, as a non-limiting example. The injector may comprise a female luer connection at one end that may be coupled to the male luer tip of a syringe or a male luer connection of an IV line, as non-limiting examples. The injector may further comprise an inner cannula that engages and pierces a seal of a connector inserted into the injector, to provide a fluid path through the injector and connector, with it recognized that the connector may be provided as a BD PhaSeal™ Optima Infusion Connector, a BD PhaSeal™ Optima Infusion Adapter, or a BD PhaSeal™ Optima Protector, as non-limiting examples. Use of an injector and connector as part of a CSTD provides substantially leak-proof sealing during engagement of multiple components, during transfer of a hazardous substance between such components, and during disengagement of the components.

While a CSTD as described is effective in providing for a safe removal of a hazardous drug from the vial and a subsequent compounding or administering of the drug, it is recognized that there is the potential for components to accidentally be disconnected during use of the CSTD. As a first example, it is possible for the female luer connection of injector to accidentally disconnect from the male luer connection of an IV line or syringe. As a second example, it is possible for the connector to become disengaged/disconnected from the injector. Disconnection of the components in this manner can result in a healthcare worker being exposed to hazardous drugs.

Accordingly, it is desirable to provide a CSTD that prevents accidentally disconnection of components therein during use. It is further desirable to provide a CSTD where the disconnection of components after use is prohibited, so as to prevent reuse of components.

### SUMMARY OF THE INVENTION

Provided herein is a closed system drug transfer device. The closed system drug transfer device comprises an injector that includes a body comprising a distal end and a proximal end, a luer hub coupled to the body at the proximal end and comprising a female luer connection and a cannula hub, a cannula retained by the cannula hub and extending distally therefrom into an inner volume of the body, and an end cap secured to the distal end of the body, the end cap comprising a lip formed thereon. The closed system drug transfer device also comprises a connector configured to be inserted into the injector through the distal end of the body. The connector includes a central shaft defining a lumen that extends between a distal end and a proximal end of the connector and a seal positioned in a proximal opening of the central shaft, the seal being pierceable by the cannula when the connector is inserted into the injector, to fluidly connect the cannula to the lumen. The central shaft comprises flanges formed on the central shaft and extending out radially therefrom and a pair of flexible fingers each comprising a proximal end attached to the central shaft and a free distal end configured to deflect radially inward and outward, with each of the flexible fingers comprising a snap protruding outward from the flexible finger at a location between the proximal and distal ends thereof and configured to engage the lip to secure the connector to the injector and a release arm positioned at the distal end of the flexible finger and extending radially outward therefrom, so as to protrude out past the snap. The release arms are engageable by a user such that, when a pinching force is applied thereto, the flexible fingers are deflected radially inward and the snaps on the flexible fingers disengage the lip, to enable removal of the connector out from the injector.

In some embodiments, the snap on each flexible finger comprises a sloped proximally-facing surface sloping radially outward from a proximal end thereof to a distal end thereof and a flat distally-facing surface that is oriented orthogonal to the central shaft, the flat distally-facing surface engaging the lip when the connector is fully inserted into the injector and preventing removal of the connector out from the injector.

In some embodiments, the radially inward deflection of the flexible fingers responsive to the pinching force being applied to the release arms causes the flat distally-facing surface of the tabs to disengage from the lip.

In some embodiments, the proximally-facing surface of each of the snaps slides along and past the lip of the end cap when the connector is inserted into the injector.

In some embodiments, the connector comprises a male luer connection provided at the distal end of the connector, extending out distally from the central shaft, the male luer connection in fluid communication with the lumen.

In some embodiments, the release arm of each of the pair of flexible fingers is spaced apart longitudinally from the male luer connection, to enable engagement of the release arms by the user and application of the pinching force thereto.

Also provided herein is another closed system drug transfer device. The closed system drug transfer device comprises an injector that includes a body comprising a distal end and a proximal end, a luer hub coupled to the body at the proximal end and comprising a female luer connection and a cannula hub, a cannula retained by the cannula hub and extending distally therefrom into an inner volume of the body, and an end cap secured to the distal end of the body, the end cap comprising a sidewall and a lip protruding radially inward from the sidewall, with the lip including a pair of slots formed therein. The closed system drug transfer device also comprises a connector configured to be inserted into the injector through the distal end of the body, the connector including a central shaft defining a lumen that extends between a distal end and a proximal end of the connector and a seal positioned in a proximal opening of the central shaft, the seal being pierceable by the cannula when the connector is inserted into the injector, to fluidly connect the cannula to the lumen. The central shaft comprises flanges formed on the central shaft and extending out radially therefrom and a pair of flexible fingers each comprising a proximal end attached to the central shaft and a free distal end configured to deflect radially inward and outward, with each of the flexible fingers comprising a snap protruding outward from the flexible finger at a location between the proximal and distal ends thereof and configured to engage the lip to secure the connector to the injector. The end cap is rotatable relative to the body and the connector between a first position and a second position, with the slots angularly aligned with the flanges when the end cap is in the first position, to enable insertion of the connector into the injector, and with the slots angularly misaligned with the flanges when the end cap is in the second position, to prevent removal of the connector out from the injector.

In some embodiments, each of the flanges comprises a projection formed thereon extending out further radially than a remainer of the flange and a notch formed distal to the projection, wherein with the connector inserted into the injector, the notch is aligned longitudinally with the lip, to enable rotation of the end cap relative to the connector.

In some embodiments, with the connector inserted into the injector and the end cap rotated to the second position, a distally-facing surface of the projection of each flange abuts the lip, to lock the connector to. the injector.

In some embodiments, the distally-facing surface of the projection of each flange is aligned longitudinally with the snaps formed on the flexible fingers.

In some embodiments, the end cap further comprises a pair of stops formed on an inner surface of the sidewall and on a proximally-facing surface of the lip, the pair of stops limiting rotation of the end cap relative to the connector, with the projections abutting the pair of stops when the end cap is rotated to the second position.

In some embodiments, the end cap comprises markings formed thereon indicating a location of the pair of slots and/or a direction of rotation of the end cap to lock the connector with the injector.

Also provided herein is another closed system drug transfer device. The closed system drug transfer device comprises a syringe comprising a male luer connection at a distal end thereof and an injector connectable to the syringe. The injector includes a body comprising a distal end and a proximal end, a luer hub rotatably coupled to the body at the proximal end and comprising a sidewall having a slot formed therein, a female luer connection engageable with the male luer connection of the syringe, and a cannula hub, a cannula retained by the cannula hub and extending distally therefrom into an inner volume of the body, a hub cover attached to the body and positioned over a portion of the luer hub to secure the luer hub on the body, with the hub cover locked in-place with the body to prevent rotation relative thereto, and with the hub cover including a sidewall comprising a slot formed therein, and a key pin having a pull tab attached thereto, with the key pin inserted through the slot of the luer hub sidewall and through the slot of the hub cover sidewall, and being removable from each of the slots responsive to a user pulling the pull tab radially away from the injector. With the key pin inserted through the slots of the luer hub sidewall and hub cover sidewall, the luer hub is locked in-place relative to the hub cover and the body such that rotation thereof is prevented, thereby enabling twisting engagement of the male luer connection to the female luer connection. With the key pin removed from the slots of the luer hub sidewall and hub cover sidewall, the luer hub freely rotates relative to the hub cover and the body, thereby preventing twisting disengagement of the male luer connection from the female luer connection.

In some embodiments, the body includes a collar formed on an outer surface thereof adjacent the proximal end, the collar including one or more notches formed therein, and wherein the hub cover includes one or more tabs formed on an inner surface of the sidewall, with the one or more tabs engaged with the one or more notches to lock the hub cover in place with the body and prevent rotation relative thereto.

In some embodiments, the hub cover comprises an open proximal end via which the female luer connection of the luer hub is accessible.

In some embodiments, with the male luer connection of the syringe connected to the female luer connection of the luer hub and the key pin removed from the slots of the luer hub sidewall and hub cover sidewall, the syringe and the luer hub are freely rotatable in each of a clockwise direction and a counterclockwise direction.

Also provided herein is another closed system drug transfer device. The closed system drug transfer device comprises a syringe including a male luer connection at a distal end thereof and an injector connectable to the syringe. The injector includes a body comprising a distal end and a proximal end, a luer hub coupled to the body at the proximal end and comprising a sidewall, a female luer connection engageable with the male luer connection of the syringe, and a cannula hub, a cannula retained by the cannula hub and extending distally therefrom into an inner volume of the body, and a locking cap attached to the luer and positioned over a portion thereof, the locking cap comprising an attachment portion securing the locking cap to the luer hub and a luer clamp pivotably connected to attachment portion, with the luer clamp including a pair of brackets movable relative to the attachment portion and relative to the female luer connection. The pair of brackets are movable from an open configuration where the brackets are pivoted away from female luer connection to a closed configuration where the brackets are connected together. With the male luer connection of the syringe connected to the female luer connection of the luer hub and the pair of brackets in the closed configuration, the luer clamp grips the male luer connection of the syringe, to prevent disconnection thereof from the female luer connection of the luer hub.

In some embodiments, each of the pair of brackets comprises a semi-circular channel defined therein that extends a length of the bracket, and wherein with the pair of brackets in the closed configuration, the semi-circular channels collectively providing an opening within which the male luer connection of the syringe may be received.

In some embodiments, the semi-circular channel includes an adhesive applied thereto that is configured to secure the luer clamp to the male luer connection of the syringe, with a peel-off cover layer applied over the adhesive that may be selectively removed therefrom to expose the adhesive.

In some embodiments, one of the pair of brackets comprises a pair of tabs and the other of the pair of brackets comprises a pair of receptacles, and wherein the pair of brackets may be secured in the closed configuration via engagement of the tabs with the receptacles.

In some embodiments, the attachment portion comprises an annular base including an opening therein within which the female luer connection is received, to provide access to the female luer connection for attachment of the syringe thereto, and a plurality of mounting arms protruding distally from the annular base, the mounting arms spaced circumferentially about the annular base and, such that the mounting arms are spaced about the sidewall of luer hub, with the plurality of mounting arms configured as flexible arms that function to provide for a snap-fit connection of the locking cap onto the luer hub.

In some embodiments, each of the pair of brackets is pivotably connected to the annular base via a living hinge component, the living hinge components enabling movement of the pair of brackets from the open configuration to the closed configuration.

Also provided herein is a non-disconnectable luer arrangement. The non-disconnectable luer arrangement comprises a male luer connector including a distal male luer connection comprising an elongated protrusion and a surrounding collar that are configured to engage a female luer connection and a proximal stem including a slot formed on an outer surface thereof, wherein in the slot, the outer surface of stem includes ratchet teeth formed thereon extending about the circumference of the stem. The non-disconnectable luer arrangement also comprises a luer cap engaged with the male luer connector, the luer cap including a cylindrical sleeve that fits about the male luer connector, with the sleeve including an open distal end and a partially closed proximal end comprising an annular flange that defines an opening therein that accommodates the stem, and with an inner surface of the annular flange that defines the opening including pawl teeth formed thereon extending about the circumference of the opening. The pawl teeth are configured to interact with the ratchet teeth to selectively provide for rotation between the luer cap and the male luer connector, with the pawl teeth and the ratchet teeth each comprising angled teeth that interact to allow for rotation of the luer cap relative to the male luer connector in one direction but prevent rotation of the luer cap relative to the male luer connector in the other direction.

In some embodiments, the annular flange fits about the slot of the stem.

In some embodiments, rotation of the luer cap relative to the male luer connector is prevented when turning the luer cap in a clockwise direction, based on a locking engagement between the pawl teeth on luer cap and the ratchet teeth on the male luer connector, thus enabling connection of the male luer connector to the female luer hub via application of a twisting motion to the luer cap, and rotation of the luer cap relative to the male luer connector is allowed when turning the luer cap in a counterclockwise direction, based on a slipping of the pawl teeth on the luer cap over the ratchet teeth on the male luer connector, thus preventing disconnection of the male luer connector from the female luer hub.

In some embodiments, with the male luer connector connected to the female luer hub, turning of the luer cap in the counterclockwise direction does not transfer rotation to the male luer connector, resulting in the male luer connector remaining engaged to female luer hub.

In some embodiments, the proximal stem defines a port configured to receive an IV line therein.

### BRIEF DESCRIPTION OF THE DRAWINGS

FIG. 1 is a perspective view of a closed-system drug transfer device (CSTD) with which embodiments of the disclosure may be implemented;

FIG. 2 is an exploded view of an injector included in the CSTD of FIG. 1;

FIG. 3 is a perspective view of additional adapters that may be included in a CSTD and with which embodiments of the disclosure may be implemented;

FIG. 4 is a side view of an injector and connector of a CSTD, according to an embodiment of the disclosure;

FIG. 5 is a side cross-sectional view of the injector and connector of FIG. 4, with the connector being inserted into the injector;

FIG. 6 is a side cross-sectional view of the injector and connector of FIG. 4, with the connector fully inserted into the injector;

FIG. 7 is a side cross-sectional view of the injector and connector of FIG. 4, with the connector being removed from the injector;

FIG. 8 is a side view of an injector and connector of a CSTD, according to another embodiment of the disclosure;

FIG. 9 is a perspective view of an end cap of the injector of FIG. 8;

FIG. 10 is a top view of the end cap of FIG. 9;

FIG. 11 is a perspective view of the connector of FIG. 8;

FIG. 12 is a side cross-sectional view of the injector and connector of FIG. 8, with the connector being inserted into the injector;

FIG. 13 is a top view showing the connector positioned relative to the end cap when being inserted into the injector;

FIG. 14 is a side cross-sectional view of the injector and connector of FIG. 8, with the connector fully inserted into the injector;

FIG. 15 is a side view of the injector and connector of FIG. 8, showing the end cap being rotated in a first direction after the connector fully is inserted into the injector, to lock the connector in the injector;

FIG. 16 is a perspective view showing the end cap being rotated relative to the connector, to lock the connector in the injector;

FIG. 17 is a side view of the injector and connector of FIG. 8, showing the end cap being rotated in a second direction after the connector fully is inserted into the injector, to unlock the connector from the injector;

FIG. 18 is a side cross-sectional view of the injector and connector of FIG. 8, with the connector being removed from the injector, after the end cap being rotated to unlock the connector from the injector;

FIG. 19 is a perspective view of an injector of a CSTD, according to another embodiment of the disclosure;

FIG. 20 is an exploded view of the injector of FIG. 19;

FIG. 21 is a perspective view of the injector of FIG. 19, showing a syringe being connected to the injector;

FIG. 22 is a perspective view of the injector of FIG. 19, showing the syringe being secured to the injector via a twisting engagement of a male luer connection of the syringe with a female luer connection of the injector;

FIG. 23 is a perspective view of the injector of FIG. 19, showing a key pin and pull tab of the injector being removed, to unlock a luer hub of the injector;

FIG. 24 is a perspective view of the injector of FIG. 19, showing the syringe and luer hub being freely rotated relative to a remainer of the injector;

FIG. 25 is a perspective view of an injector of a CSTD, with a luer clamp thereof in an open configuration, according to another embodiment of the disclosure;

FIG. 26 is a perspective view of the injector of FIG. 25, with the luer clamp in a closed configuration;

FIG. 27 is a perspective view of the injector of FIG. 25, showing a syringe being connected to the injector;

FIG. 28 is a perspective view of the injector of FIG. 25, showing the syringe being secured to the injector via a twisting engagement of a male luer connection of the syringe with a female luer connection of the injector;

FIG. 29 is a perspective view of the injector of FIG. 25, showing the luer clamp being actuated from the open configuration to the closed configuration, to secure the syringe to the injector;

FIG. 30 is a perspective view of a non-disconnectable luer arrangement, according to another embodiment of the disclosure;

FIG. 31 is a cross-sectional perspective view of the non-disconnectable luer arrangement of FIG. 30;

FIG. 32 is a cross-sectional side view of the non-disconnectable luer arrangement of FIG. 30, with a male luer connector exploded out from a luer cap;

FIG. 33 is a cross-sectional side view of the non-disconnectable luer arrangement of FIG. 30, with the male luer connector secured in the luer cap;

FIG. 34 is a cross-sectional side view of the non-disconnectable luer arrangement of FIG. 30 being initially engaged with a female luer connection of an injector;

FIG. 35 is a partial perspective view of the non-disconnectable luer arrangement of FIG. 30, showing rotation of the luer cap in a clockwise direction, to facilitate engagement of the non-disconnectable luer arrangement with the female luer connection;

FIG. 36 is a cross-sectional side view of the non-disconnectable luer arrangement of FIG. 30 fully engaged with the female luer connection of the injector; and

FIG. 37 is a partial perspective view of the non-disconnectable luer arrangement of FIG. 30, showing rotation of the luer cap in a counterclockwise direction, with luer cap slipping over the male luer connector, such that the non-disconnectable luer arrangement remains engaged with the female luer connection.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with another component or with a patient. Thus, for example, the end of a device first contacting another component or the body 44 of the patient would be the distal end, while the opposite end of the device being manipulated by the user would be the proximal end of the device.

The terms “first”, “second”, and the like are not intended to refer to any particular order or chronology, but refer to different conditions, properties, or elements.

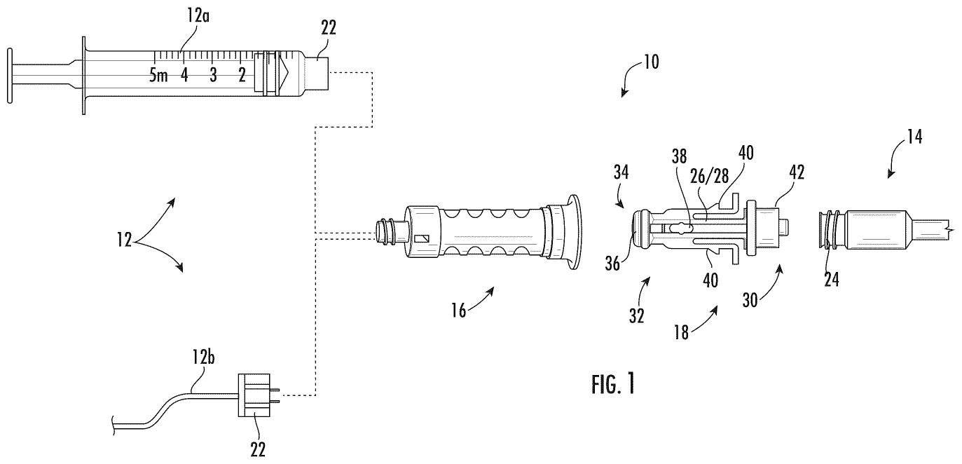

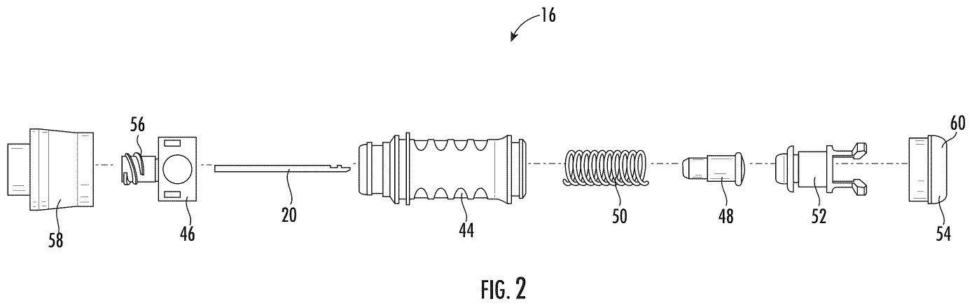

Referring to FIGS. 1 and 2, shown is a non-limiting embodiment of a system 10 for the closed transfer of fluids (i.e., a closed-system drug transfer device (CSTD) 10) with which aspects of the disclosure may implemented. In the illustrated embodiment, the CSTD 10 may provide for the closed transfer of fluids from/between a first fluid source, container, or conduit 12 and a second fluid source, container, or conduit 14. The CSTD 10 includes an injector 16 and a connector 18. The CSTD 10 provides substantially leak-proof sealing during engagement of a cannula 20 of the injector 16 with connector 18, during transfer of a substance from/between the first fluid source, container, or conduit 12 and the second fluid source, container, or conduit 14 that flows through the injector 16 and connector 18. The leak-proof sealing of the CSTD 10 substantially prevents leakage of both air and liquid during use of the CSTD 10.

In the non-limiting embodiment of FIG. 1, the first fluid source, container, or conduit 12 may be provided as a syringe 12a or intravenous (IV) line 12b that is connected to a proximal end of the injector 16. The syringe 12a or IV line 12b comprises a male luer connection 22 thereon that enables a mechanical and fluid connection with the injector 16. Also, in the non-limiting embodiment of FIG. 1, the second fluid source, container, or conduit 14 may be provided as an adapter or fluid conduit that is connected to a distal end of connector 18. The adapter or fluid conduit comprises a female luer connection 24 thereon that enables a mechanical and fluid connection with the connector 18.

As shown in FIG. 1, the connector 18 is configured to be secured to each of the injector 16 and the female luer connection 24 of the second fluid source, container, or conduit 14, to form an open fluid path therebetween. The connector 18 includes a central shaft 26 defining a lumen 28 that extends between a distal end 30 and a proximal end 32 of the connector 18. The lumen 28 ends at a proximal opening 34 provided at the proximal end 32 of the central shaft 26, and a self-closing or self-healing seal 36 is provided over the proximal opening 34 that may be pierced by a cannula 20 of the injector 16 (FIG. 2) to provide access to the lumen 28 when the injector 16 is connected to connector 18. The central shaft 26 may include an arrangement of flanges 38 and flexible fingers 40 that protrude radially outward therefrom and that mate with the injector 16 when the injector 16 is slid linearly into the connector 18. That is, while not shown in FIG. 1, it is recognized that the flanges 38 and flexible fingers 40 would engage with guiding grooves formed on an interior surface of the injector 16, with the snap fit fingers 40 flexing and forming a snap-fit engagement with a lip of the injector 16 to secure the connector 18 in the injector 16.

According to the embodiment of FIG. 1, a male luer connection 42 is provided at the distal end 30 of the central shaft 26. The male luer connection 42 comprises an elongated protrusion and a surrounding collar and is configured to engage the female luer connection 24 of the second fluid source, container, or conduit 14, to mechanically and fluid connect the connector 18 thereto.

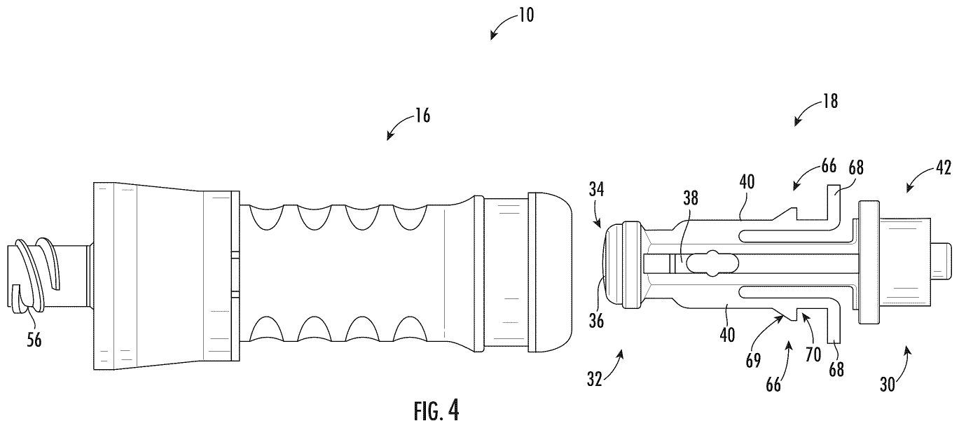

As best shown in FIG. 2, the injector 16 includes a body 44, a luer hub 46, a septum 48, a spring 50, a connector clip 52, a (distal) end cap 54, and the cannula 20. The luer hub 46 is secured to the body 44 at a proximal end of the injector 16 and includes a female luer connection 56 configured to engage the male luer connection 22 of the syringe 12a or IV line 12b. The luer hub 46 may comprise a threaded outer surface, as well as a tapered luer cavity, with the cavity retaining the septum 48 therein. The luer hub 46 may also include a cannula hub 57 (see FIGS. 5-7, for example) therein within which cannula 20 may be secured, with the cannula 20 extending out distally from the cannula hub and into a hollow interior of body 44. In some embodiments, the injector 16 may also include a hub cover 58 that may be secured to the proximal end of body 44 and that functions to secure the luer hub 46 on the body 44. The spring 50 is secured within the hollow interior of body 44, at the proximal end of the body 44. The connector clip 52 is secured to a distal end of the spring 50 and is positioned within the hollow interior of body 44. The connector clip 52 is configured to engage the proximal end of connector 18 when the connector 18 is pressed into engagement with injector 16, with the connector 18 engaging a shoulder of connector clip 52 when the connector 18 is fully engaged with injector 16. With the connector 18 fully engaged with injector 16, the cannula 20 penetrates through the seal 36 of connector 18, such that the cannula 20 is brought into fluid communication with the lumen 28 of connector 18-thereby forming a continuous fluid path extending from the luer hub 46 of injector 16, through connector 18, and to the female luer connection 24 of the second fluid source, container, or conduit 14, such that the first fluid source, container, or conduit 12 is fluidly connected to the second fluid source, container, or conduit 14 and a transfer of fluid therebetween is provided. The end cap 54 is formed integrally with or secured to the distal end of body 44 and functions to maintain engagement between the connector 18 and the injector 16 when the connector 18 is pressed into engagement with injector 16-with a lip 60 on end cap 54 engaging the flexible fingers 40 to secure the connector 18 to injector 16.

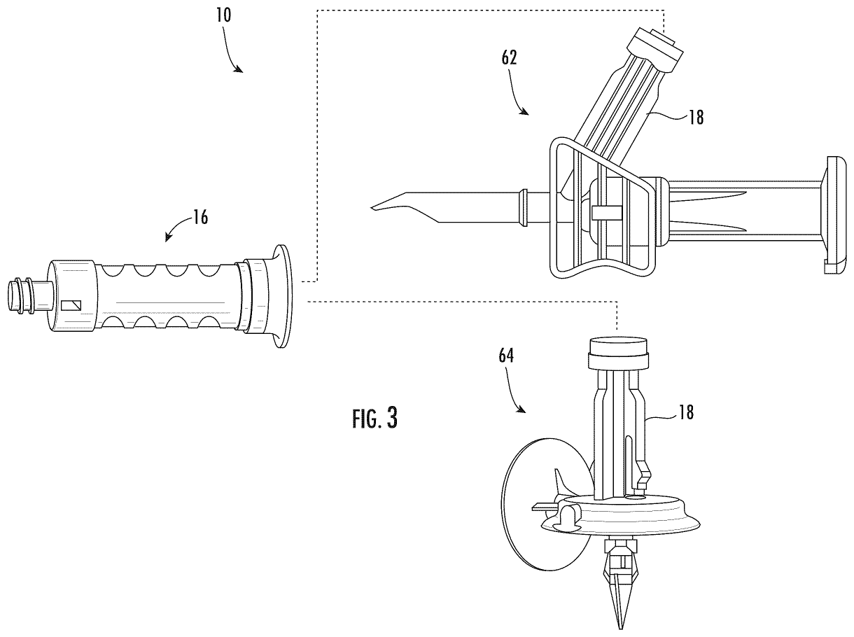

While the connector 18 of the CSTD 10 of FIG. 1 is shown as a stand-alone connector/component that connects to each of the injector 16 and the female luer connection 24 of the second fluid source, container, or conduit 14, it is recognized that the connector 18 could instead be integrated into and form part of a larger component used in the CSTD 10. Referring to FIG. 3, additional embodiments are illustrated where the connector 18 forms part of a fluid infusion adapter 62 that may be used for puncturing/accessing an IV bag or forms part of a vial adapter or protector 64 that may be used for puncturing/accessing a vial. In either embodiment, the connector 18 would still be configured to engage the injector 16 and provide for a closed transfer of a fluid through the injector 16. Still other embodiments are envisioned where a CSTD 10 is compatible to be used with IV bags, IV lines, patient connectors, or other aspects to move fluids between fluid sources, containers or conduits.

It is recognized that there is the potential for components of the CSTD 10 to disconnect from one another or for the CSTD 10 to disconnect from the syringe 12a or IV line 12b during use of the CSTD 10. As a first example, it is possible for the injector 16 to accidentally disconnect from the syringe 12a or IV line 12b, such as via the male luer connection 22 of the syringe 12 a or IV line 12b unscrewing from the female luer connection 56 of luer hub 46. As a second example, it is possible for the connector 18 to accidentally disconnect from the injector 16, such as when a pulling force is applied to the injector 16, for example. Such a disconnection of the syringe 12a/IV line 12b and/or connector 18 from the injector 16 can result in a healthcare worker being exposed to hazardous drugs.

According to aspects of the disclosure, a CSTD 10 may be provided that is configured to prevent accidental disconnection between the connector 18 and the injector 16.

Referring to FIGS. 4-7, a CSTD 10 is provided that is configured to prevent accidental disconnection between the connector 18 and the injector 16. The connector 18 and the injector 16 are configured similar to the connector 18 and the injector 16 shown and described in FIGS. 1 and 2, except that the connector 18 includes features thereon that provide a controlled connection and disconnection from injector 16.

The connector 18 includes a central shaft 26 defining a lumen 28 that extends between a distal end 30 and a proximal end 32 of the connector 18. The lumen 28 ends at a proximal opening 34 provided at the proximal end 32 of the central shaft 26, and a seal 36 is provided over the proximal opening 34 that may be pierced by the cannula 20 of the injector 16 (FIGS. 5-7) to provide access to the lumen 28 when the injector 16 is connected to connector 18. The central shaft 26 may include an arrangement of flanges 38 and flexible fingers 40 that protrude radially outward therefrom and that mate with the injector 16 when the injector 16 is slid linearly downward onto the connector 18—with the flanges 38 arranged perpendicular to the flexible fingers 40. According to one embodiment, a male luer connection 42 is provided at the distal end 30 of the central shaft 26. The male luer connection 42 comprises an elongated protrusion and a surrounding collar.

According to embodiments of the disclosure, each of the flexible fingers 40 includes a snap 66 and a release arm 68 thereon that function to provide for engagement and disengagement, respectively, of the connector 18 to/from the injector 16.

The snaps 66 are formed on flexible fingers 40 between proximal and distal ends of the fingers 40 and at a location that provides for engagement between the snaps 66 with the lip 60 formed on the end cap 54 of injector 16. Each of the snaps 66 includes a sloped proximally-facing surface 69 and a flat distally-facing surface 70 that is orthogonal to the elongated central shaft 26. The sloped proximally-facing surface 69 of snaps 66 allows for the connector 18 to be pushed proximally into the injector 16, with the proximally-facing surface 69 sliding along and past the lip 60 of the end cap 54 of injector 16 (with the fingers 40 deflecting radially inward as the snaps 66 slide past lip 60 and deflecting back radially outward after the snaps 66 have moved past lip 60). Upon the connector 18 being fully pushed into injector 16, the flat distally-facing surface 70 of snaps 66 functions to lock the connector 18 in the injector 16—with a pulling out of the connector 18 from the injector 16 in the distal direction being prevented due to the flat distally-facing surface 70 abutting/engaging the lip 60.

The release arms 68 are formed on flexible fingers 40 at the distal end thereof and are formed/bent so as to extend radially outward from the remainder of the flexible fingers 40. The release arms 68 are spaced apart longitudinally from the male luer connection 42 of connector 18 and from the snaps 66. The release arms 68 are formed such that they extend out radially past the snaps 66 and the collar of male luer connection 42, and the release arms 68 also extend out radially past the end cap 54 of injector 16, in order that the release arms 68 may be engaged by a healthcare provider. In use of the CSTD 10, the healthcare provider may apply a pinching force to the release arms 68 in order to cause the flexible fingers 40 to deflect radially inward, such that the snaps 66 become disengaged from the lip 60 of end cap 54—thereby allowing the connector 18 to be separated from the injector 16 when desired.

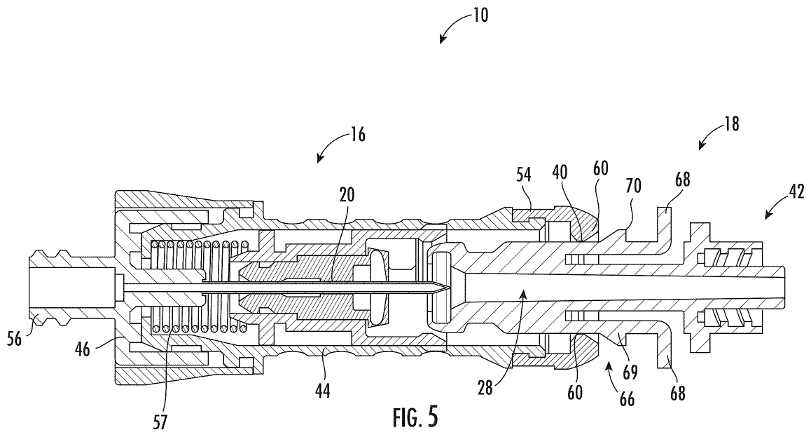

Referring now to FIGS. 5-7, connection and disconnection of the connector 18 and injector 16 is illustrated. As shown first in FIG. 5, the connector 18 and injector 16 are joined by pushing the connector 18 and injector 16 into engagement with each other. As the connector 18 and injector 16 are pushed into engagement, the flexible fingers 40 slide along lip 60 of end cap 54. When the sloped proximally-facing surface 69 of snaps 66 are brought into contact with lip 60, the flexible fingers 40 are caused to deflect radially inward as the proximally-facing surface 69 of snaps 66 slide along and past the lip 60. Once the proximally-facing surface 69 of snaps 66 moves proximally past the lip 60, the flexible fingers 40 deflect back radially outward, with the flat distally-facing surface 70 of snaps 66 thus brought into engagement with a proximally-facing surface 69 of lip 60 and thereby locking the connector 18 in engagement with the injector 16, as shown in FIG. 6. In this arrangement, a pulling out of the connector 18 from the injector 16 in the distal direction is prevented due to the flat distally-facing surface 70 abutting/engaging the lip 60 and locking the connector 18 in place.

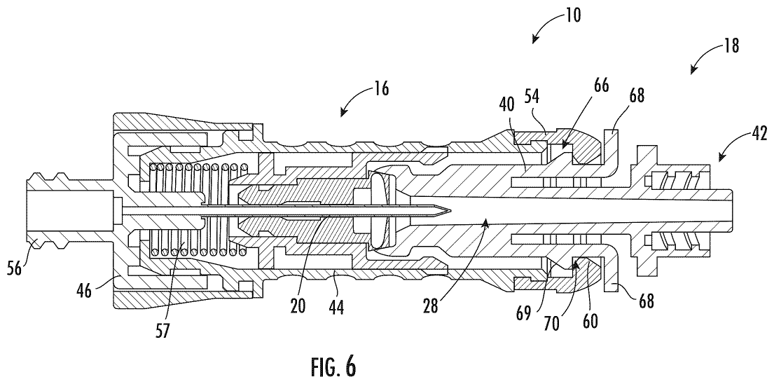

As shown in FIG. 7, when it is desired to separate the connector 18 and the injector 16, a healthcare provider applies a pinching force to the release arms 68 in order to cause the flexible fingers 40 to deflect radially inward, such that the snaps 66 become disengaged from the lip 60 of end cap 54—i.e., the snaps 66 are moved radially inward from lip 60. With the snaps 66 separated from engagement with lip 60, the healthcare provider may then pull the connector 18 distally out from the injector 16.

Accordingly, with the connector 18 formed to include flexible fingers 40 having snaps 66 and release arms 68, accidental disengagement of the connector 18 from the injector 16 is prevented (due to the flat distally-facing surface 70 of snaps 66 locking with lip 60), while allowing for selective disengagement of the connector 18 from the injector 16 when desired (via application of a pinching force to release arms 68).

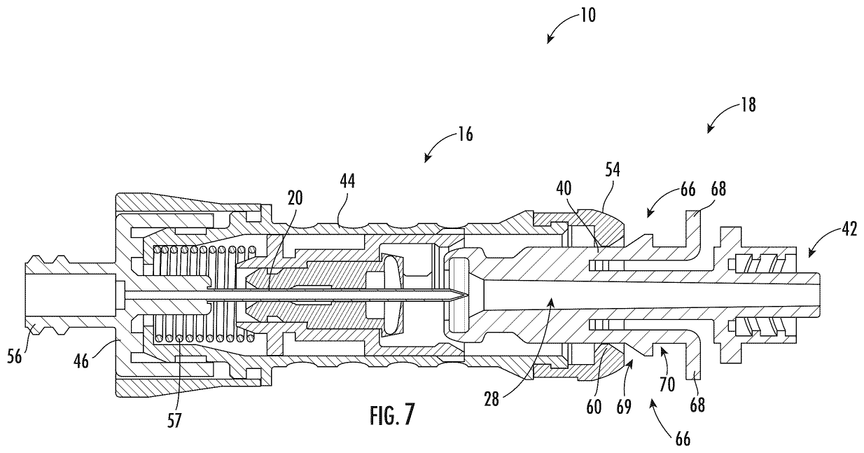

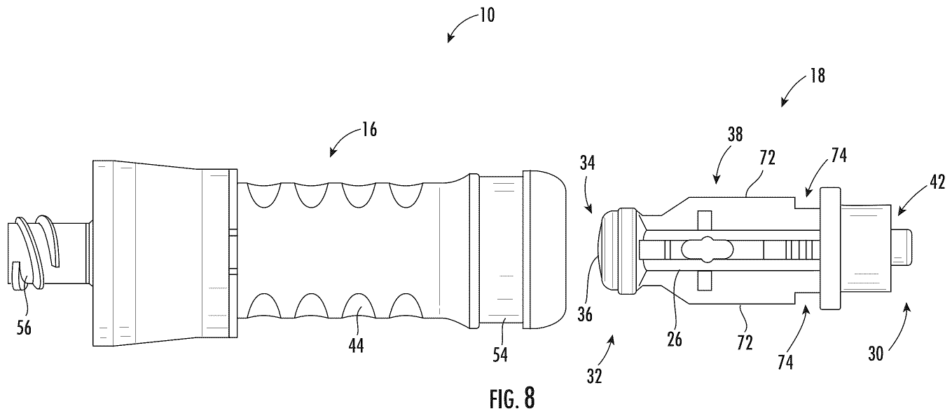

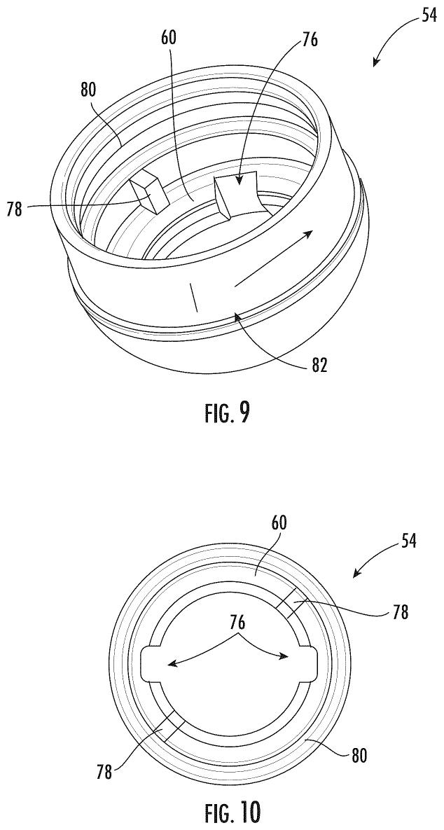

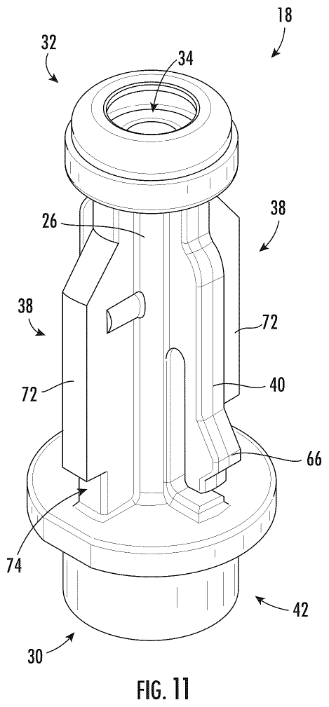

Referring now to FIGS. 8-18, another CSTD 10 is provided that is configured to prevent accidental disconnection between the connector 18 and the injector 16. The connector 18 and the injector 16 are configured similar to the connector 18 and the injector 16 shown and described in FIGS. 1-7, except each of the injector 16 and the connector 18 includes features thereon that provide a controlled connection and disconnection from injector 16.

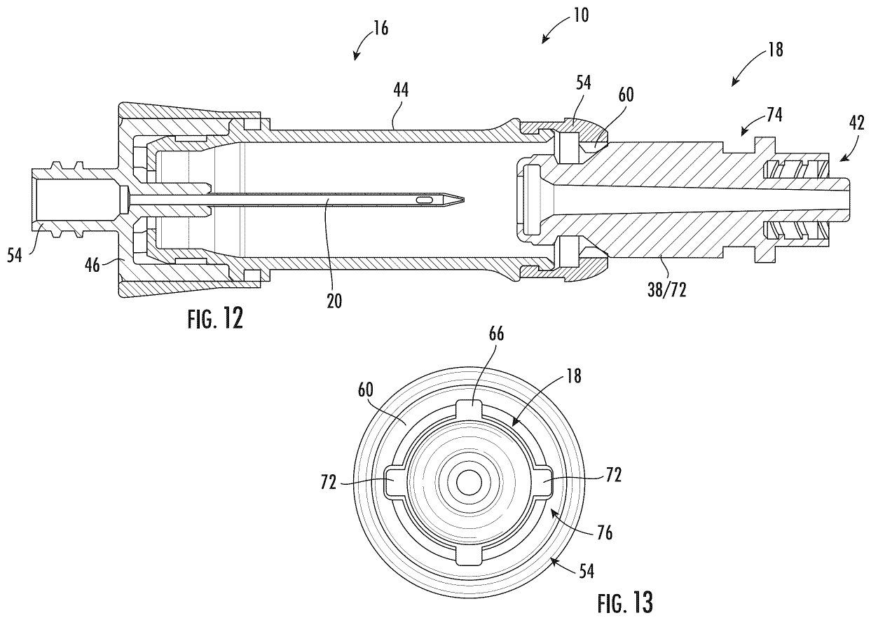

The connector 18 includes a central shaft 26 defining a lumen 28 that extends between a distal end 30 and a proximal end 32 of the connector 18. The lumen 28 ends at a proximal opening 34 provided at the proximal end 32 of the central shaft 26, and a seal 36 is provided over the proximal opening 34 that may be pierced by a cannula 20 of the injector 16 (see FIGS. 12 and 14) to provide access to the lumen 28 when the injector 16 is connected to connector 18. The central shaft 26 may include an arrangement of flanges 38 and flexible fingers 40 that protrude radially outward therefrom and that mate with the injector 16 when the injector 16 is slid linearly downward onto the connector 18-with a pair of flanges 38 provided on opposing side of the central shaft 26 and arranged perpendicular to a pair of flexible fingers 40. According to one embodiment, a male luer connection 42 is provided at the distal end 30 of the central shaft 26. The male luer connection 42 comprises an elongated protrusion and a surrounding collar.

According to embodiments of the disclosure, each of the flanges 38 is formed to include a projection 72 thereon that increases a radial width of the flange 38 along a portion of the length thereof, with the projection 72 on the flange 38 extending out radially further than the flexible fingers 40. Each of the flanges 38 is further formed to include a notch or cut-out 74 formed at the distal end thereof (i.e., distal to projection 72), such that the far distal end of the flanges 38 is inset radially inward from the projections 72 thereof. In some embodiments, a bottom end of the projections 72 on flanges 38 is aligned lengthwise/longitudinally with snaps 74 formed on the flexible fingers 40—with the snaps 74 have sloped proximally-facing and distally-facing surfaces.

In connection with the structure of the flanges 38 of connector 18, the end cap 54 of injector 16 is specifically designed to accommodate the flanges 38. That is, end cap 54 of injector 16 is configured to include a pair of slots 76 formed in the lip 60—with the slots 76 formed in opposing sides of the lip 60. The slots 76 extend through an entirety of a thickness of the lip 60 and are sized to accommodate passage of the flanges 38 (i.e., of projections 72 of flanges 38) therethrough when engaging the connector 18 with injector 16. Also provided on end cap 54 are a pair of stops 78 formed on an inner surface of a sidewall 80 of the end cap 54 and on a proximal-facing surface of the lip 60. The pair of stops 78 are formed on opposing sides of the sidewall 80 and protrude radially inward therefrom, with the stops 78 functioning to limit rotation between the connector 18 and the injector 16, as explained in further detail below.

In some embodiments, markings 82 may be provided on the injector 16 (i.e., on end cap 54) indicating the location of slots 76 and/or indicating a direction to rotate the end cap 54 relative to connector 18 (to bring stops 78 into contact with flange projections 72).

Referring now to FIGS. 12-18, connection and disconnection of the connector 18 and injector 16 is illustrated. As shown first in FIGS. 12 and 13, joining of the connector 18 and injector 16 is initiated by first angularly aligning the slots 76 of end cap 54 with the flanges 38 of connector 18 by rotating the end cap 54 on injector body 44. Upon aligning of the slots 76 with flanges 38, the connector 18 is then pushed into engagement with injector 16, as shown in FIG. 14. As the connector 18 and injector 16 are pushed into engagement, the projections 72 of flanges 38 are allowed to pass through the slots 76, while snaps 66 on the flexible fingers 40 slide along and past the lip 60 of end cap 54. When the snaps 66 are pushed past the lip 60 and the projections 72 of flanges 38 are positioned proximal to the lip 60, the connector 18 is fully engaged with the injector 16.

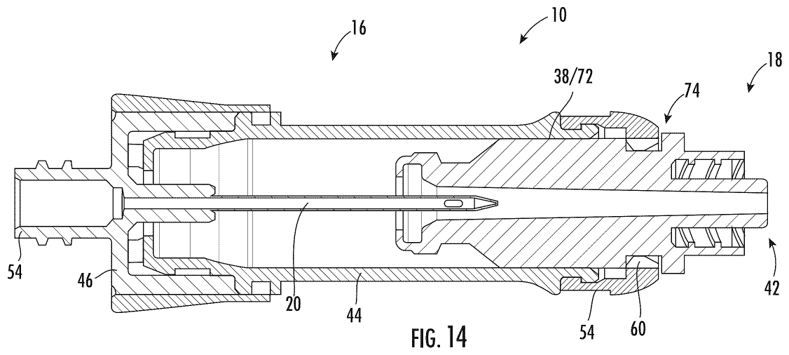

Upon the connector 18 being fully engaged with the injector 16, the end cap 54 is then rotated relative to the connector 18, as shown in FIGS. 15 and 16, such that the slots 76 are angularly misaligned with the flange projections 72 of connector 18. According to embodiments, the end cap 54 may be rotated until the projections 72 of flanges 38 abut the stops 78 formed on end cap 54, with the notches 74 formed in flanges 38 allowing for such rotation of the end cap 54 (and lip 60 thereof) relative to flange projections 72. With the flanges 38 angularly misaligned from the slots 76, a pulling out of the connector 18 from the injector 16 in the distal direction is prevented due to interference and locking of the projections 72 with the lip 60—i.e., the projections 72 of flanges 38 abut the lip 60 and prevent disconnection of the connector 18 from injector 16.

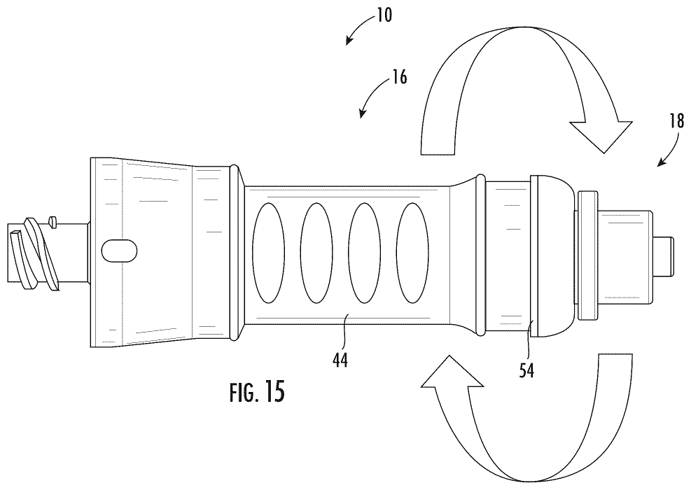

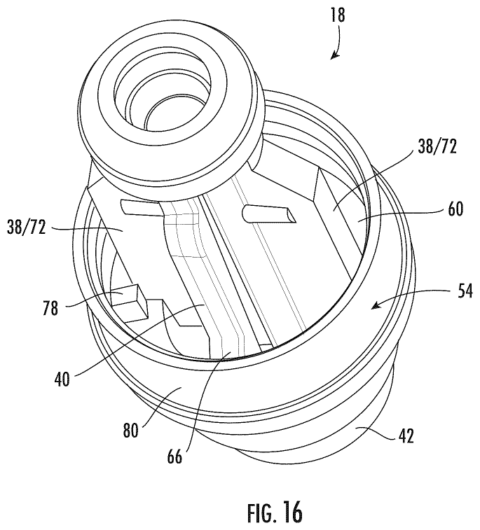

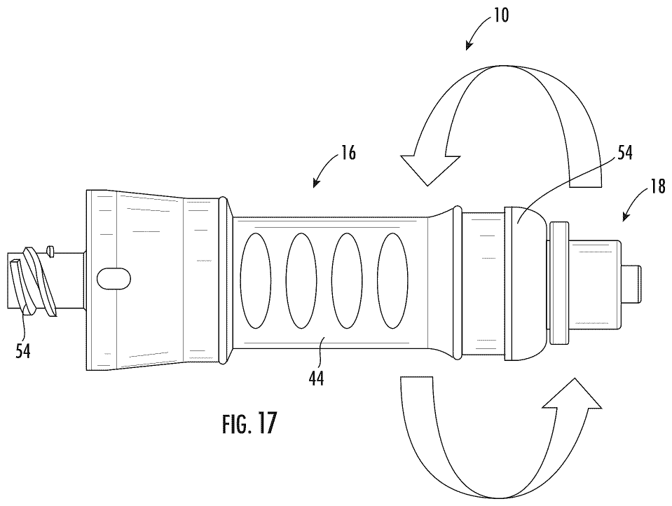

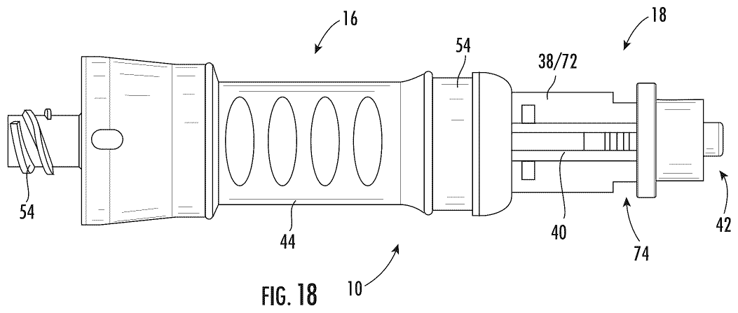

Referring now to FIGS. 17 and 18, in order to disconnect the connector 18 from injector 16, the end cap 54 of injector 16 is rotated in the opposite direction until the slots 76 in lip 60 are realigned with the flange projections 72. With the slots 76 realigned with the projections 72, a healthcare provider can then pull the connector 18 distally out from the injector 16, with the projections 72 sliding out through the slots 76 and the flexible fingers 40 deflecting radially inward so that snaps 66 slide past lip 60.

Accordingly, with the connector 18 formed to include flanges 38 with radial projections 72 thereon and the injector 16 formed to include an end cap 54 with slots 76 formed therein, accidental disengagement of the connector 18 from the injector 16 is prevented when the end cap 54 is rotated relative to the connector 18 (due to the projections 72 locking with lip 60), while allowing for selective disengagement of the connector 18 from the injector 16 when desired by rotating the end cap 54 back relative to the connector 18 (to realign the slots 76 with projections 72).

According to additional aspects of the disclosure, a CSTD may be provided that is configured to prevent accidental disconnection between the injector 16 and the syringe 12a or IV line 12b (hereafter referred to generally as “syringe 12a”).

Referring to FIGS. 19-24, a CSTD 10 is provided that is configured to prevent accidental disconnection between the injector 16 and the male luer connection 22 of the syringe 12a. The injector 16 may be configured similar to the injector 16 shown and described in any of FIGS. 1-18, except that the injector 16 includes features thereon that prevent disconnection of the male luer connection 22 of the syringe 12a from injector 16.

The injector 16 includes a body 84, a luer hub 86, a hub cover 88, and a pull tab 90 and removable key pin 92. The luer hub 86 is secured to the body 84 at the proximal end of the connector 18 and includes a female luer connection 94 configured to engage the male luer connection 22 of the syringe 12a. The hub cover 88 may be secured to the proximal end of body 84 and function to secure the luer hub 86 on the body 84. While not shown, it is also understood that injector 16 also includes a septum 48, a spring 50, a connector clip 52, an end cap 54, and a cannula 20, as shown and described in previous embodiments.

According to aspects of the disclosure, the luer hub 86, hub cover 88, body 84, and pull tab 90 and removable key pin 92 are constructed and assembled so as to provide a non-detachable luer connection between the syringe and luer hub 86, so as to prevent reuse of the CSTD, while also allowing the luer hub 86 to selectively rotate after connection of the syringe to ensure clear visibility of syringe markings without entangling intravenous lines. In particular, by selectively allowing and preventing rotation of the luer hub 86, connection of the syringe to the luer hub 86 is enabled, while a subsequent disconnection of the syringe from the luer hub 86 may be prevented.

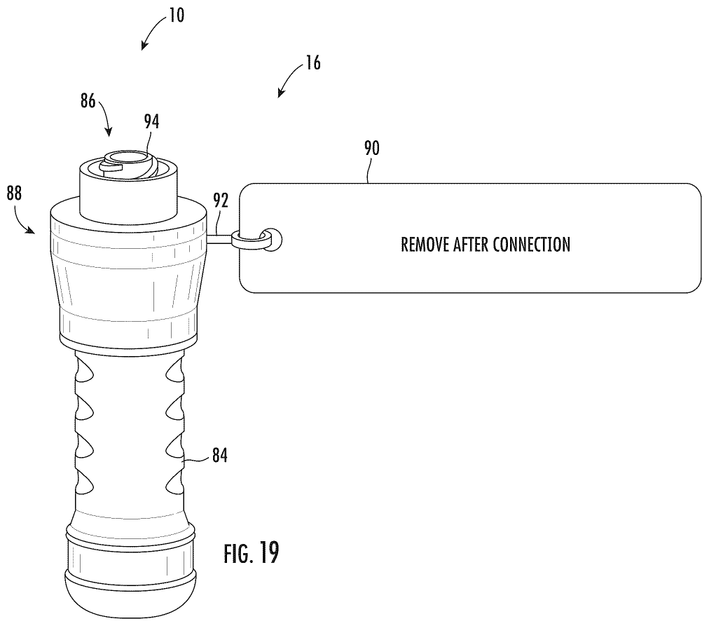

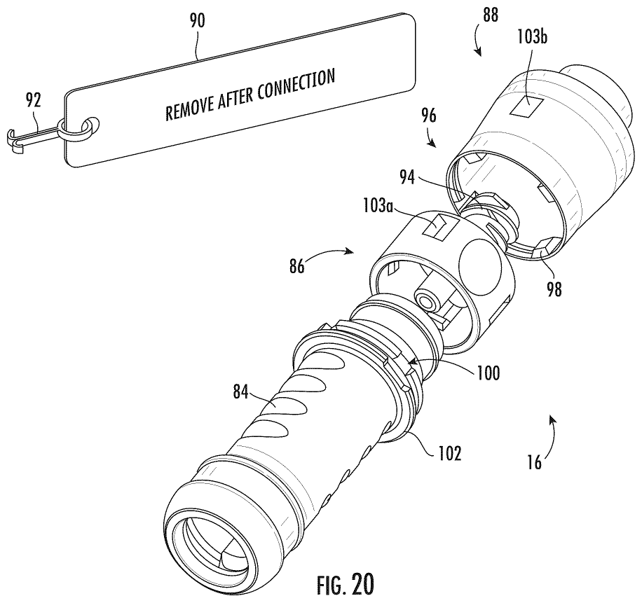

As best shown in FIG. 20, the injector 16 is assembled so that luer hub 86 is rotatably coupled to body 84 at the proximal end of body 84, with the luer hub 86 retained on body 84 by hub cover 88. The hub cover 88 secures the luer hub 86 on body 84, while still providing access to the female luer connection 94 of luer hub 86 via a proximal open end 96 of the hub cover 88. The hub cover 88 may be locked in position relative to body 84 via engagement of one or more tabs 98 formed on an inner circumferential surface of hub cover 88 with one or more notches 100 formed in an outer collar 102 of body 84—with the tab(s) 98 engaged in notch(es) 100 to prevent rotation of the hub cover 88 relative to the body 84.

According to embodiments of the disclosure, rotation of the luer hub 86 relative to body 84 (and relative to hub cover 88) is selectively allowed or prevented based on the inclusion of key pin 92 of injector 16. That is, each of luer hub 86 and hub cover 88 include a slot 103a, 103b formed in a sidewall thereof (with the slots 103a, 103b initially aligned with one another) within which key pin 92 may be inserted-with the key pin 92 preventing rotation of the luer hub 86 relative to hub cover 88 (and body 84) when the key pin 92 is inserted through the slots 103a, 103b. The key pin 92 may be selectively removed from slots 103a, 103b by application of a pulling force to the pull tab 90 that is connected to key pin 92 and, upon removal of the key pin 92, the luer hub 86 is then free to rotate relative to hub cover 88 and body 84.

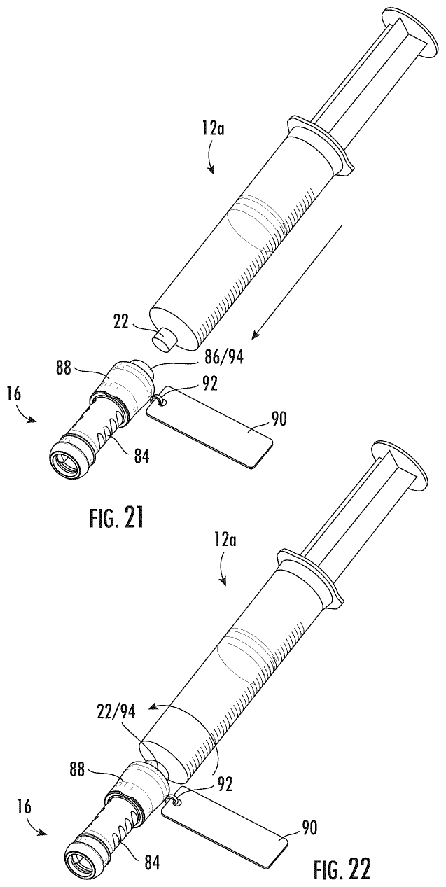

Referring now to FIGS. 21-24, connection and usage of the syringe 12a with injector 16 is illustrated. As shown first in FIGS. 21 and 22, with the injector 16 in an initial state where removable key pin 92 is joined to hub cover 88 and luer hub 86 (i.e., key pin 92 is inserted through slots 103a, 103b thereof), rotation of the luer hub 86 relative to the hub cover 88 and body 84 is prevented-as the key pin 92 prevents any rotation of luer hub 86 due to its insertion within slots 103a, 103b. With the luer hub 86 in a locked configuration, the syringe 12a may be connected to luer hub 86, with a twisting engagement of the male luer connection 22 of syringe 12a and the female luer connection 94 of luer hub 86 enabled based on the locked configuration of luer hub 86.

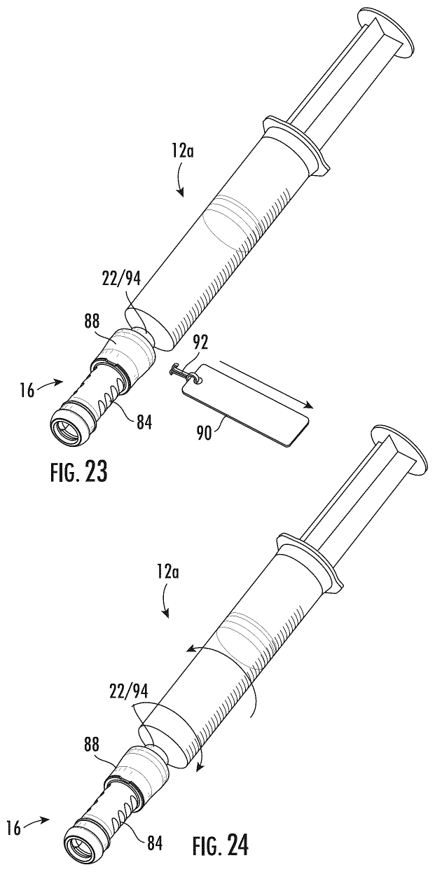

After the syringe 12a has been secured to injector 16 vis twisting engagement of the male luer connection 22 of syringe 12a and the female luer connection 94 of luer hub 86, the key pin 92 may then be removed via a pulling of the pull tab 90, as shown in FIG. 23. As described above, removal of the key pin 92 from the hub cover 88 and luer hub 86 (i.e., removal of key pin 92 from slots 103a, 103b) serves to “disengage” the luer hub 86 from hub cover 88, such that rotation of the luer hub 86 relative to body 84 (and relative to hub cover 88) is enabled-while the hub cover 88 remains locked in place relative to body 84 based on engagement of tab(s) 98 with notch(es) 100. With the luer hub 86 rotatable relative to body 84 (and relative to hub cover 88), any rotation/twisting of the syringe will not result in disconnection of the syringe from the luer hub 86, but instead the luer hub 86 will freely rotate along with the syringe, as shown in FIG. 24. Accordingly, with the luer hub 86 disengaged from hub cover 88, the syringe 12a and luer hub 86 are able to freely rotate in both clockwise (CW) and counterclockwise (CCW) directions. This free bi-directional rotation of the syringe 12a ensures clear visibility of syringe markings without entangling intravenous lines, while also preventing disconnection of the syringe 12a from injector 16 (i.e., prevents both accidental and intentional disconnection). With a healthcare provider being prevented from disconnecting the syringe 12a from injector 16, re-use of the injector 16 is prevented-ensuring single-use integrity of the injector 16.

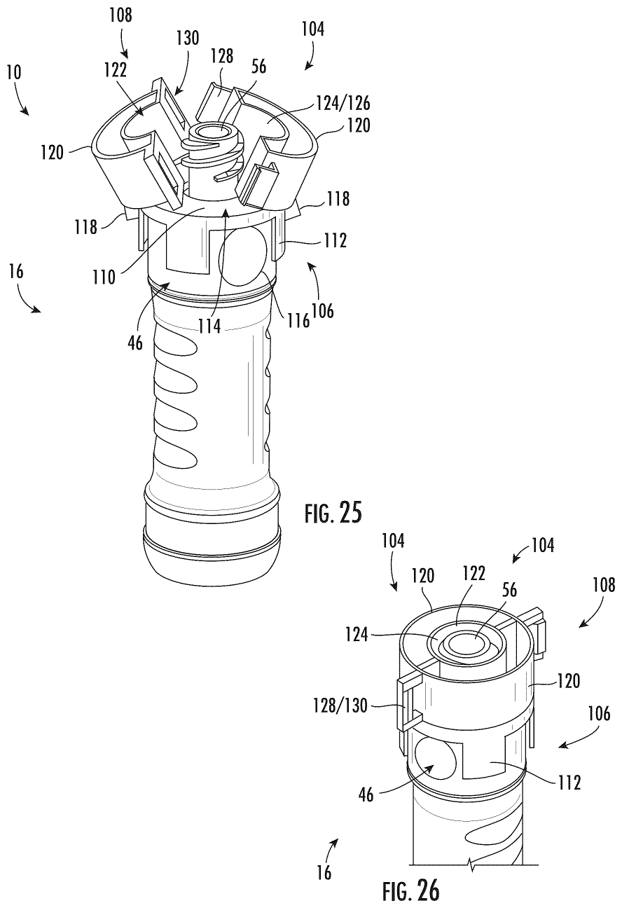

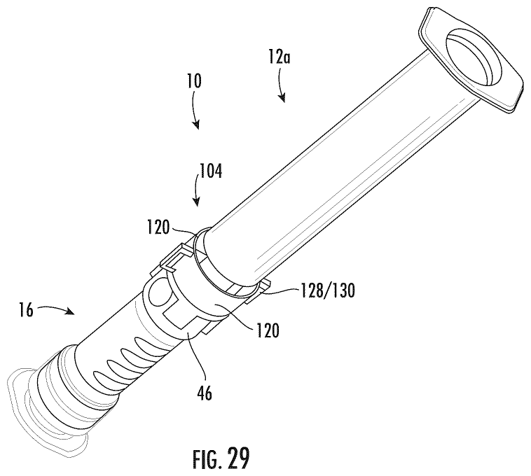

Referring to FIGS. 25-29, another CSTD 10 is provided that is configured to prevent accidental disconnection between the injector 16 and the syringe 12. The connector 18 and the injector 16 are configured similar to the connector 18 and the injector 16 shown and described in FIGS. 1-18, except that a locking cap 104 is further added to injector 16 that prevents disconnection of the syringe 12a from injector 16.

According to aspects of the disclosure, the locking cap 104 is secured to the proximal end of injector 16 and functions to secure a syringe 12a (or IV line 12b, in other embodiments) to the luer hub 46, so as to prevent accidental disconnection of the syringe 12a from the injector 16. The locking cap 104 may generally comprise an attachment portion 106 and a luer clamp 108, with the attachment portion 106 securing the locking cap 104 to luer hub 46 and the luer clamp 108 operable to secure the syringe 12a to luer hub 46.

The attachment portion 106 of locking cap 104 may comprise an annular base 110 and a plurality of mounting arms 112 protruding distally from the annular base 110. The annular base 110 is positioned over the luer hub 46, with an opening 114 in the annular base 110 receiving the female luer connection 56 of luer hub 46 therein and allowing access to the female luer connection 56, for attachment of the syringe 12a thereto. The mounting arms 112 are spaced circumferentially about the annular base 110 and protrude down distally therefrom, such that the mounting arms 112 are spaced about a sidewall 116 of luer hub 46 when the locking cap 104 is positioned over the luer hub 46. The mounting arms 112 are configured as flexible arms that function to provide for a snap-fit connection of the locking cap 104 onto the luer hub 46.

The luer clamp 108 is pivotably connected to attachment portion 106, such as via a living hinge connection or interface 118, with the luer clamp 108 positioned on a proximal-facing surface of the annular base 110. The luer clamp 108 is formed of two brackets 120 (e.g., semi-cylindrical brackets) that are positioned on annular base 110 so as to be on opposing sides of the female luer connection 56 of luer hub 46. Each of the brackets 120 is pivotably connected to the annular base 110 via a living hinge component 118, such that the brackets 120 are movable/pivotable relative to annular base 110 and relative to female luer connection 56. Each bracket 120 of the luer clamp 108 may comprise a C-shaped bracket that defines a semi-circular channel 122 formed therein, through which the female luer connection 56 may pass. As shown best in FIG. 25, the channel 122 of each bracket includes an adhesive 124 applied thereto that aids in securing the luer clamp 108 to the male luer connection 22 of syringe 12a after the syringe is connected to luer hub 46. A peel-off cover layer 126 may initially be provided over the adhesive 124 and may be removed once the syringe 12a has been connected to luer hub 46. According to embodiments, any of a number of suitable adhesives may be applied in the channels 122, with acrylate including methacrylate and epoxy diacrylates (which are also known as vinyl resins) being a non-limiting example.

The brackets 120 of luer clamp 108 may be locked together via interacting tabs 128 and receptacles 130 configured to engage each other upon the brackets 120 being moved into contact with each other. A user may push the tabs 128 of one bracket 120 into the receptacles 130 of the other bracket 120 upon positioning of the luer clamp 108 about the male luer connection 22 of syringe 12a and female luer connection 56 of luer hub 46 (after syringe 12a is connected to luer hub 46), to thus lock the luer clamp 108 in a closed configuration.

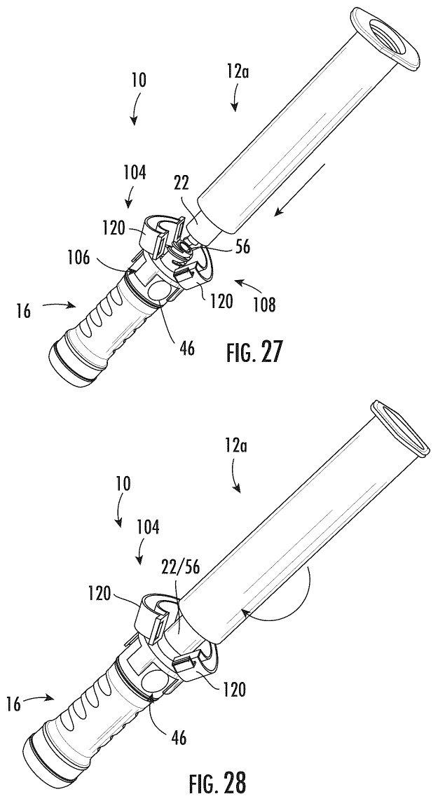

Referring now to FIGS. 27-29, connection and usage of the syringe 12a with injector 16 is illustrated. Initially, it is recognized that the locking cap 104 may first be attached to the injector 16 (i.e., to luer hub 46), with the locking cap 104 pressed onto the proximal end of injector 16 such that mounting arms 112 snap into engagement with luer hub 46 and secure the locking cap 104 thereto, as shown in FIG. 27. When initially attaching the locking cap 104 to luer hub 46, the brackets 120 are in a spread or open configuration and pivoted away from female luer connection 56, so as to provide easy access to the female luer connection 56.

With the locking cap 104 attached to luer hub 46, the syringe may be connected to luer hub 46 as shown in FIG. 28, with a twisting engagement of the male luer connection 22 of syringe 12a and the female luer connection 56 of luer hub 46 securing the syringe 12a to the injector 16.

Once the syringe 12a is secured to injector 16, the peel-off cover 126 on each of brackets 120 may be removed, so as to expose the adhesive-covered channel 122 in each of the brackets 120. Once the peel-off covers 126 on the brackets 120 are removed and the adessive 124 is exposed, the brackets 120 are then pivoted into engagement with each other-with the tabs 128 of one bracket 120 being pushed into the receptacles 130 of the other bracket 120 to secure the brackets 120 together in a closed configuration. With the brackets 120 in the closed configuration, the adhesive 124 in the channels 122 of the brackets 120 is brought into contact with the outer collar of the male luer connection 22 of syringe 12a—such that the syringe 12a is firmly gripped by the locking cap 104. With the syringe 12a gripped by the locking cap 104 and the locking cap 104 attached to luer hub 46, the syringe 12a and injector 16 are secured together and the syringe 12a is prevented from disconnecting from injector 16 (i.e., male luer connection 22 is preventing from unscrewing from female luer connection 56).

Referring now to FIGS. 31-38, a non-disconnectable luer arrangement 132 is provided that is configured to prevent disconnection between a male luer connector 134 (such as may be found on an IV line 12b) and an injector 16 of a CSTD 10. The injector 16 may be configured similar to the injector 16 shown and described in FIGS. 1-17, with the injector 16 including a luer hub 46 having a female luer connection 56 thereon. As explained in detail below, a luer cap 136 may be added to a male luer connector 134 and may be configured to enable connection of the male luer connector 134 to the female luer connection 56 (of luer hub 46) but prevent a subsequent disconnection of the male luer connector 134 from the female luer connection 56, which may be desirable to prevent possible spilling or leakage of hazardous drugs.

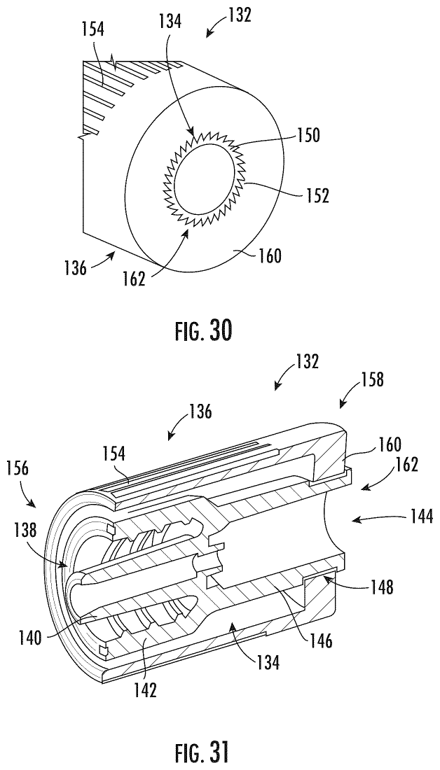

As shown in FIGS. 30-37, the non-disconnectable luer arrangement 132 includes a male luer connector 134 having a distal end that includes male luer connection 138 comprising an elongated protrusion 140 and a surrounding collar 142 that are configured to engage a female luer connection, such as the female luer connection 56 on luer hub 46 of injector 16. In some embodiments, the opposing proximal end of the male luer connector 134 may comprise a port 144 configured to receive an IV line therein-with the IV line being pressure fit into the port 144 to provide a secure connection between the male luer connector 134 and the IV line. The port 144 may be defined by a proximal stem 146 of the male luer connector 134, with the proximal stem 146 extending out and away from the collar 142 and protrusion 140. The stem 146 is configured to include a slot 148 formed on an outer surface thereof via which the luer cap 136 may be secured to the male luer connector 134. Additionally, in the region of slot 148, the outer surface of stem 146 includes ratchet teeth 150 formed thereon extending about the circumference of the stem 146, with the ratchet teeth 150 provided to interact with pawl teeth 152 formed on the luer cap 136 to selectively provide for rotation between the luer cap 136 and the male luer connector 134, as explained in further detail below.

The luer cap 136 may be configured as a cylindrical sleeve 154 that fits about the male luer connector. The sleeve 154 of luer cap 136 includes an open distal end 156 and a partially closed proximal end 158-with the partially closed proximal end 158 comprising an annular flange 160 that defines a hole or opening 162 therein that accommodates the stem 146 of male luer connector. An inner edge/surface of the annular flange 160 that defines opening 162 includes pawl teeth 152 formed thereon extending about the circumference of the opening 162, with the pawl teeth 152 configured to interact with the ratchet teeth 150 formed on the stem 146 of male luer connector to selectively provide for rotation between the luer cap 136 and the male luer connector. Specifically, the pawl teeth 152 on luer cap 136 and the ratchet teeth 150 on the male luer connector 134 each comprise angled teeth that interact to allow for rotation of the luer cap 136 relative to the male luer connector 134 in one direction but prevent rotation of the luer cap 136 relative to the male luer connector 134 in the other direction.

With engagement of the luer cap 136 and male luer connector 134 as described above including interaction between the pawl teeth 152 on luer cap 136 and the ratchet teeth 150 on the male luer connector 134-the luer cap 136 is configured to allow for connection of the male luer connector 134 with a female luer connection 56 but prevent a subsequent disconnection of the male luer connector 134 from the female luer connection 56. That is, a rotation of the luer cap 136 relative to the male luer connector 134 may be prevented when grasping and turning the luer cap 136 in the clockwise direction based on a locking engagement between the pawl teeth 152 on luer cap 136 and the ratchet teeth 150 on the male luer connector 134, which allows for the male luer connector 134 (and luer cap 136 thereon) to be connected to a female luer connection 56 (such as on injector 16) via application of a twisting force/motion to luer cap 136. Conversely, a rotation of the luer cap 136 relative to the male luer connector 134 may be allowed when grasping and turning the luer cap 136 in the counterclockwise direction based on a slipping of the pawl teeth 152 on luer cap 136 over the ratchet teeth 150 on the male luer connector 134, which prevents disconnection of the male luer connector 134 from the female luer connection 56, as any rotation of luer cap 136 is not transferred to male luer connector 134-resulting in the male luer connector 134 remaining engaged/secured to female luer connection 56.

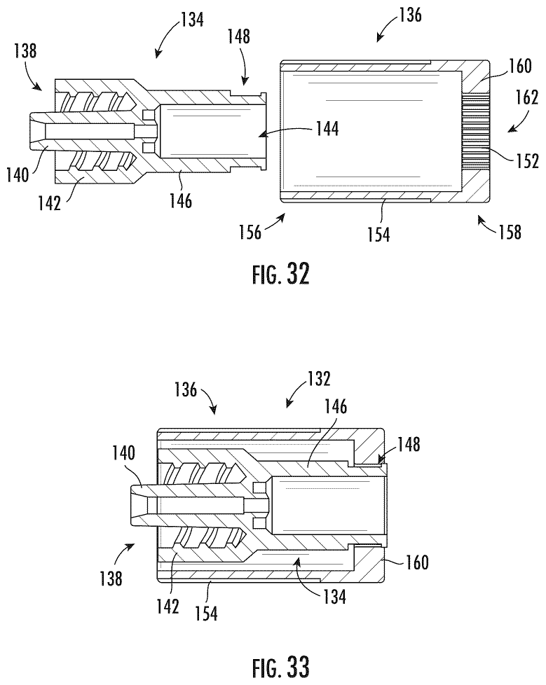

Operation and connection of non-disconnectable luer arrangement 132 is now described in FIGS. 33-37.

As shown in FIG. 333, the non-disconnectable luer arrangement 132 is shown as initially assembled, with the luer cap 136 secured on male luer connector 134.

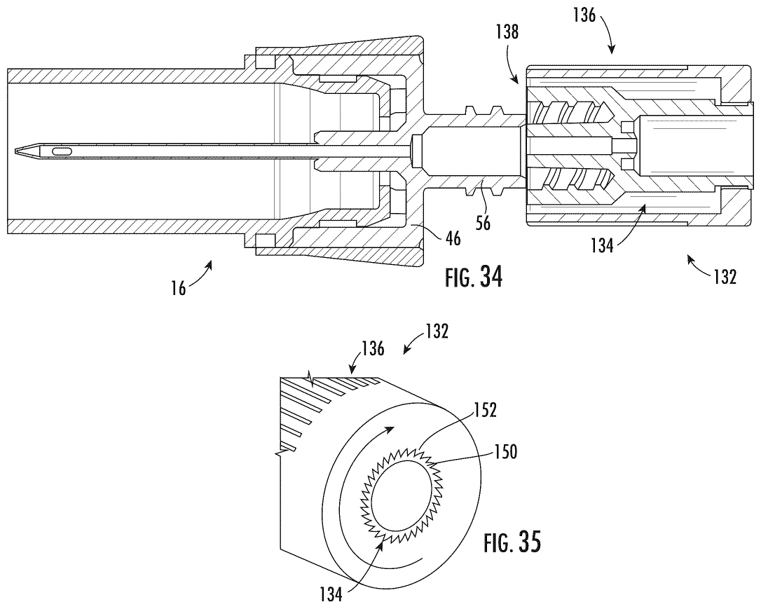

As shown in FIGS. 34 and 35, the non-disconnectable luer arrangement 132 is shown being connected to a female luer connection 56, such as provided on the luer hub 46 of an injector 16. In connecting the non-disconnectable luer arrangement 132 to female luer connection 56, a twisting force/motion is applied to the luer cap 136 in the clockwise direction, with a locking engagement between the pawl teeth 152 on luer cap 136 and the ratchet teeth 150 on the male luer connector 134 transmitting the twisting motion to the male luer connector 134 to enable connection thereof to the female luer connection 56.

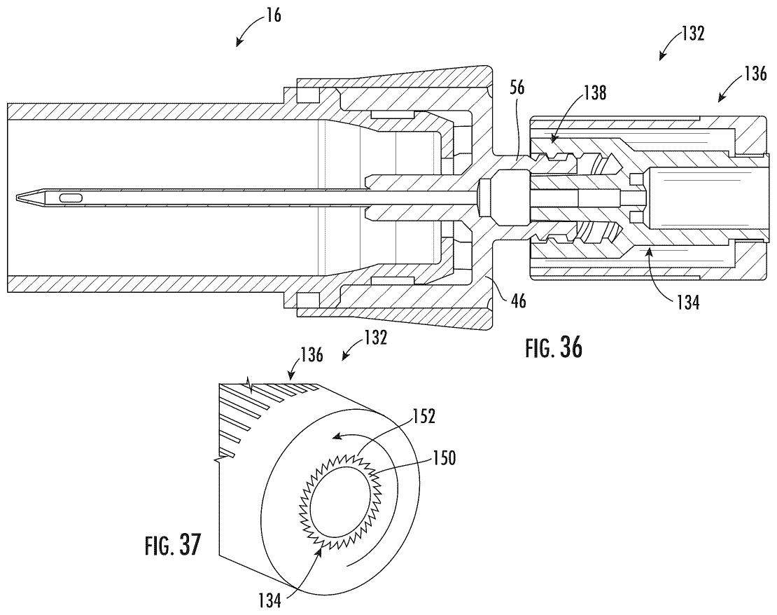

Upon connection of the male luer connector 134 to the female luer connection 56, as shown in FIG. 36, a twisting force/motion may be applied to the luer cap 136 in the counterclockwise direction, as shown in FIG. 37. However, due to a slipping of the pawl teeth 152 on luer cap 136 over the ratchet teeth 150 on the male luer connector 134 that occurs responsive to the counterclockwise twisting force/motion, the luer cap 136 rotates relative to the male luer connector 134, which prevents disconnection of the male luer connector 134 from the female luer connection 56, as any rotation of luer cap 136 is not transferred to male luer connector 134-resulting in the male luer connector 134 remaining engaged/secured to female luer connection 56.

Beneficially, aspects of the disclosure described herein provide a CSTD that is configured to prevent accidental disconnection between a connector and the injector thereof and/or is configured to prevent accidental or intentional disconnection between the injector and the male luer connection of a syringe or IV line. The controlled connect and disconnect features of the CSTD help to prevent leaks of a hazardous drug in the CSTD and may also prevent the disconnection of components after use thereof, so as to prevent reuse of the CSTD.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A closed system drug transfer device comprising:
an injector including:
a body comprising a distal end and a proximal end;
a luer hub coupled to the body at the proximal end, the luer hub comprising a female luer connection and a cannula hub;
a cannula retained by the cannula hub and extending distally therefrom into an inner volume of the body; and
an end cap secured to the distal end of the body, the end cap comprising a lip formed thereon; and
a connector configured to be inserted into the injector through the distal end of the body, the connector including:
a central shaft defining a lumen that extends between a distal end and a proximal end of the connector; and
a seal positioned in a proximal opening of the central shaft, the seal being pierceable by the cannula when the connector is inserted into the injector, to fluidly connect the cannula to the lumen;
wherein the central shaft comprises:
flanges formed on the central shaft and extending out radially therefrom; and
a pair of flexible fingers each comprising a proximal end attached to the central shaft and a free distal end configured to deflect radially inward and outward, with each of the flexible fingers comprising:
a snap protruding outward from the flexible finger at a location between the proximal and distal ends thereof and configured to engage the lip to secure the connector to the injector; and
a release arm positioned at the distal end of the flexible finger and extending radially outward therefrom, so as to protrude out past the snap;
wherein the release arms are engageable by a user such that, when a pinching force is applied thereto, the flexible fingers are deflected radially inward and the snaps on the flexible fingers disengage the lip, to enable removal of the connector out from the injector.

2. The device of claim 1, wherein the snap on each flexible finger comprises:
a sloped proximally-facing surface sloping radially outward from a proximal end thereof to a distal end thereof; and
a flat distally-facing surface that is oriented orthogonal to the central shaft, the flat distally-facing surface engaging the lip when the connector is fully inserted into the injector and preventing removal of the connector out from the injector.

3. The device of claim 2, wherein the radially inward deflection of the flexible fingers responsive to the pinching force being applied to the release arms causes the flat distally-facing surface of the tabs to disengage from the lip.

4. The device of claim 2, wherein the proximally-facing surface of each of the snaps slides along and past the lip of the end cap when the connector is inserted into the injector.

5. The device of claim 1, wherein the connector comprises a male luer connection provided at the distal end of the connector, extending out distally from the central shaft, the male luer connection in fluid communication with the lumen.

6. The device of claim 5, wherein the release arm of each of the pair of flexible fingers is spaced apart longitudinally from the male luer connection, to enable engagement of the release arms by the user and application of the pinching force thereto.

7. A closed system drug transfer device comprising:
an injector including:
a body comprising a distal end and a proximal end;
a luer hub coupled to the body at the proximal end, the luer hub comprising a female luer connection and a cannula hub;
a cannula retained by the cannula hub and extending distally therefrom into an inner volume of the body; and
an end cap secured to the distal end of the body, the end cap comprising a sidewall and a lip protruding radially inward from the sidewall, with the lip including a pair of slots formed therein; and
a connector configured to be inserted into the injector through the distal end of the body, the connector including:
a central shaft defining a lumen that extends between a distal end and a proximal end of the connector; and
a seal positioned in a proximal opening of the central shaft, the seal being pierceable by the cannula when the connector is inserted into the injector, to fluidly connect the cannula to the lumen;
wherein the central shaft comprises:
flanges formed on the central shaft and extending out radially therefrom; and
a pair of flexible fingers each comprising a proximal end attached to the central shaft and a free distal end configured to deflect radially inward and outward, with each of the flexible fingers comprising a snap protruding outward from the flexible finger at a location between the proximal and distal ends thereof and configured to engage the lip to secure the connector to the injector;
wherein the end cap is rotatable relative to the body and the connector between a first position and a second position, with the slots angularly aligned with the flanges when the end cap is in the first position, to enable insertion of the connector into the injector, and with the slots angularly misaligned with the flanges when the end cap is in the second position, to prevent removal of the connector out from the injector.

8. The device of claim 7, wherein each of the flanges comprises:
a projection formed thereon extending out further radially than a remainer of the flange; and
a notch formed distal to the projection;
wherein with the connector inserted into the injector, the notch is aligned longitudinally with the lip, to enable rotation of the end cap relative to the connector.

9. The device of claim 7, wherein with the connector inserted into the injector and the end cap rotated to the second position, a distally-facing surface of the projection of each flange abuts the lip, to lock the connector to. the injector.

10. The device of claim 9, wherein the distally-facing surface of the projection of each flange is aligned longitudinally with the snaps formed on the flexible fingers.

11. The device of claim 7, wherein the end cap further comprises a pair of stops formed on an inner surface of the sidewall and on a proximally-facing surface of the lip, the pair of stops limiting rotation of the end cap relative to the connector, with the projections abutting the pair of stops when the end cap is rotated to the second position.

12. The device of claim 7, wherein the end cap comprises markings formed thereon indicating a location of the pair of slots and/or a direction of rotation of the end cap to lock the connector with the injector.

13. A closed system drug transfer device comprising:
a syringe comprising a male luer connection at a distal end thereof; and
an injector connectable to the syringe, the injector including:
a body comprising a distal end and a proximal end;
a luer hub rotatably coupled to the body at the proximal end, the luer hub comprising a sidewall having a slot formed therein, a female luer connection engageable with the male luer connection of the syringe, and a cannula hub;
a cannula retained by the cannula hub and extending distally therefrom into an inner volume of the body;
a hub cover attached to the body and positioned over a portion of the luer hub to secure the luer hub on the body, with the hub cover locked in-place with the body to prevent rotation relative thereto, and with the hub cover including a sidewall comprising a slot formed therein; and
a key pin having a pull tab attached thereto, with the key pin inserted through the slot of the luer hub sidewall and through the slot of the hub cover sidewall, and being removable from each of the slots responsive to a user pulling the pull tab radially away from the injector;
wherein with the key pin inserted through the slots of the luer hub sidewall and hub cover sidewall, the luer hub is locked in-place relative to the hub cover and the body such that rotation thereof is prevented, thereby enabling twisting engagement of the male luer connection to the female luer connection; and
wherein with the key pin removed from the slots of the luer hub sidewall and hub cover sidewall, the luer hub freely rotates relative to the hub cover and the body, thereby preventing twisting disengagement of the male luer connection from the female luer connection.

14. The device of claim 13, wherein the body includes a collar formed on an outer surface thereof adjacent the proximal end, the collar including one or more notches formed therein, and wherein the hub cover includes one or more tabs formed on an inner surface of the sidewall, with the one or more tabs engaged with the one or more notches to lock the hub cover in place with the body and prevent rotation relative thereto.

15. The device of claim 14, wherein the hub cover comprises an open proximal end via which the female luer connection of the luer hub is accessible.

16. The device of claim 15, wherein with the male luer connection of the syringe connected to the female luer connection of the luer hub and the key pin removed from the slots of the luer hub sidewall and hub cover sidewall, the syringe and the luer hub are freely rotatable in each of a clockwise direction and a counterclockwise direction.

17. A closed system drug transfer device comprising:
a syringe comprising a male luer connection at a distal end thereof; and
an injector connectable to the syringe, the injector including:
a body comprising a distal end and a proximal end;
a luer hub coupled to the body at the proximal end, the luer hub comprising a sidewall, a female luer connection engageable with the male luer connection of the syringe, and a cannula hub;
a cannula retained by the cannula hub and extending distally therefrom into an inner volume of the body; and
a locking cap attached to the luer and positioned over a portion thereof, the locking cap comprising:
an attachment portion securing the locking cap to the luer hub; and
a luer clamp pivotably connected to attachment portion, with the luer clamp including a pair of brackets movable relative to the attachment portion and relative to the female luer connection;
wherein the pair of brackets are movable from an open configuration where the brackets are pivoted away from female luer connection to a closed configuration where the brackets are connected together; and
wherein with the male luer connection of the syringe connected to the female luer connection of the luer hub and the pair of brackets in the closed configuration, the luer clamp grips the male luer connection of the syringe, to prevent disconnection thereof from the female luer connection of the luer hub.

18. The device of claim 17, wherein each of the pair of brackets comprises a semi-circular channel defined therein that extends a length of the bracket, and wherein with the pair of brackets in the closed configuration, the semi-circular channels collectively providing an opening within which the male luer connection of the syringe may be received.

19. The device of claim 18, wherein the semi-circular channel includes an adhesive applied thereto that is configured to secure the luer clamp to the male luer connection of the syringe, with a peel-off cover layer applied over the adhesive that may be selectively removed therefrom to expose the adhesive.

20. The device of claim 17, wherein one of the pair of brackets comprises a pair of tabs and the other of the pair of brackets comprises a pair of receptacles, and wherein the pair of brackets may be secured in the closed configuration via engagement of the tabs with the receptacles.

21. The device of claim 17, wherein the attachment portion comprises:
an annular base including an opening therein within which the female luer connection is received, to provide access to the female luer connection for attachment of the syringe thereto; and
a plurality of mounting arms protruding distally from the annular base, the mounting arms spaced circumferentially about the annular base and, such that the mounting arms are spaced about the sidewall of luer hub, with the plurality of mounting arms configured as flexible arms that function to provide for a snap-fit connection of the locking cap onto the luer hub.

22. The device of claim 17, wherein each of the pair of brackets is pivotably connected to the annular base via a living hinge component, the living hinge components enabling movement of the pair of brackets from the open configuration to the closed configuration.

23. A non-disconnectable luer arrangement comprising:
a male luer connector including:
a distal male luer connection comprising an elongated protrusion and a surrounding collar that are configured to engage a female luer connection; and
a proximal stem including a slot formed on an outer surface thereof, wherein in the slot, the outer surface of stem includes ratchet teeth formed thereon extending about the circumference of the stem; and
a luer cap engaged with the male luer connector, the luer cap comprising a cylindrical sleeve that fits about the male luer connector, with the sleeve including an open distal end and a partially closed proximal end comprising an annular flange that defines an opening therein that accommodates the stem, and with an inner surface of the annular flange that defines the opening including pawl teeth formed thereon extending about the circumference of the opening;
wherein the pawl teeth are configured to interact with the ratchet teeth to selectively provide for rotation between the luer cap and the male luer connector, with the pawl teeth and the ratchet teeth each comprising angled teeth that interact to allow for rotation of the luer cap relative to the male luer connector in one direction but prevent rotation of the luer cap relative to the male luer connector in the other direction.

24. The non-disconnectable luer arrangement of claim 23, wherein the annular flange fits about the slot of the stem.

25. The non-disconnectable luer arrangement of claim 23, wherein rotation of the luer cap relative to the male luer connector is prevented when turning the luer cap in a clockwise direction, based on a locking engagement between the pawl teeth on luer cap and the ratchet teeth on the male luer connector, thus enabling connection of the male luer connector to the female luer hub via application of a twisting motion to the luer cap; and
wherein rotation of the luer cap relative to the male luer connector is allowed when turning the luer cap in a counterclockwise direction, based on a slipping of the pawl teeth on the luer cap over the ratchet teeth on the male luer connector, thus preventing disconnection of the male luer connector from the female luer hub.

26. The non-disconnectable luer arrangement of claim 23, wherein with the male luer connector connected to the female luer hub, turning of the luer cap in the counterclockwise direction does not transfer rotation to the male luer connector, resulting in the male luer connector remaining engaged to female luer hub.

27. The non-disconnectable luer arrangement of claim 23, wherein the proximal stem defines a port configured to receive an IV line therein.
